(19) United States
(12) Patent Application Publication
(10) Pub. No.: US 2025/0262459 A1
(43) Pub. Date: Aug. 21, 2025

(54) LABYRINTH-TYPE NEUTRON BEAMFORMING APPARATUS FOR NEUTRON CAPTURE THERAPY

(71) Applicant: KOREA INSTITUTE OF RADIOLOGICAL & MEDICAL SCIENCES, Seoul (KR)

(72) Inventors: Bong Hwan HONG, Seoul (KR); Min Ho KIM, Seoul (KR); Seung Woo PARK, Seoul (KR); Cha Won PARK, Hwaseong-si (KR)

(21) Appl. No.: 18/702,955

(22) PCT Filed: Nov. 16, 2022
(86) PCT No.: PCT/KR2022/018101

§ 371 Date: Apr. 19, 2024

(30) Foreign Application Priority Data
Nov. 30, 2021 (KR) 10-2021-0169024

(51) Int. Cl. A61N 5/10; G21K 1/06; G21K 5/04

(52) U.S. Cl. CPC A61N 5/1077; G21K 1/067; G21K 5/04; A61N 2005/109; A61N 2005/1094

(57) ABSTRACT

The present invention relates to a labyrinth-type neutron beamforming apparatus for neutron capture therapy that can maintain a neutron flux and dramatically reduce a gamma ray flux by changing a path of the beamforming apparatus like a labyrinth and setting the neutron flux at various angles from the reflective surface and the axis in the front-and-rear direction.

### CROSS-REFERENCE TO RELATED APPLICATIONS

This application is the National Stage filing under 35 U.S.C. 371 of International Application No. PCT/KR2022/018101, filed on Nov. 16, 2022, which claims the benefit of Korean Patent Application No. 10-2021-0169024 filed on Nov. 30, 2021, the contents of which are all hereby incorporated by reference herein in their entirety.

### TECHNICAL FIELD

The present disclosure relates to a labyrinth-type neutron beamforming apparatus for neutron capture therapy

### BACKGROUND ART

Boron neutron capture therapy is a treatment method that injects a material containing boron in advance to accumulate boron in cancer cells, then irradiates neutrons to cause nuclear fission within the cancer cells, and kills the cancer cells by releasing particles due to nuclear fission. Boron neutron capture therapy is known to be effective for brain tumors, head and neck cancer, and skin cancer, and is attracting attention as a next-generation cancer treatment method because it can minimize side effects caused by radiation exposure of normal cells compared to conventional radiation treatment methods.

Neutrons generated by the boron neutron capture treatment device are classified into fast neutrons with an energy of 10 keV or more, epithermal neutrons with an energy of 0.5 eV to 10 keV, and thermal neutrons with an energy of 0.5 eV or less. It is divided into Among these, fast neutrons have high penetrating power and cause radioactive side effects in tissues surrounding the tumor, while thermal neutrons with low energy cause radioactive side effects on the skin and are not suitable for therapeutic purposes.

US Patent No. U.S. Ser. No. 10/124,192 is disclosed in relation to this boron neutron capture device. Meanwhile, in this prior art, fast neutron filters, gamma ray filters, etc. were used by arranging them in the beam direction to control unnecessary doses from radiation that may occur during neutron capture treatment. The use of these filters had the disadvantage of reducing the flux of epithermal neutrons needed for treatment.

### DISCLOSURE

### Technical Problem

To solve problems with the conventional beamforming apparatus used in the boron neutron capture therapy device, the present disclosure provides a beamforming apparatus that minimizes the reduction of neutron flux and at the same time minimizes the dose contamination caused by gamma rays and fast neutrons.

### Technical Solution

As a means of solving the above problem, a neutron beam shaping module is provided along the direction of movement of neutrons and is configured to reduce gamma rays and fast neutron flux and minimize the reduction of epithermal neutron flux, and is provided surrounding the circumference of the neutron beam shaping module. A labyrinth-type neutron beamforming apparatus for neutron capture therapy may be provided, including a reflector, wherein the neutron beam shaping module is provided along an axis from the front to the back, and at least a portion of the module is provided along a path different from the axis.

An aspect of the above module, the neutron beam shaping module is configured to shield fast neutrons, and is provided in a first shield disposed adjacent to the target for neutron generation and at the rear end of the first shield, and is configured to reduce the energy of neutrons. It may be configured to include a moderator and a second shielding unit provided at a rear end of the moderator and configured to shield thermal neutrons and gamma rays.

Additionally, the inclined surface may be formed at the boundary of at least one of the first shield, the moderator, and the second shield.

Additionally, the reflecting surfaces provided on the left and right sides of the neutron beam shaping module may be provided asymmetrically.

Meanwhile, the inclined surface is formed at the interface between the moderator and the reflector, and may be configured to have at least two different angles.

Meanwhile, two different inclined surfaces may be arranged at positive and negative angles from a plane parallel to the direction of movement of the particle beam.

Additionally, the interface between the moderator and the reflector may include a reflecting surface provided at an angle parallel to the irradiation direction of the particle beam.

Meanwhile, the first shielding part and the second shielding part may be provided parallel to each other.

Additionally, the centers of the first shield and the second shield may be located on the irradiation path of the particle beam.

Meanwhile, the centers of the first shield and the second shield may be located at a different point from the irradiation path of the particle beam.

Additionally, the first shielding part and the second shielding part may be provided at different angles.

Meanwhile, the first shielding agent may be arranged so as not to be perpendicular to the irradiation direction of the particle beam.

### Advantageous Effects

The labyrinth-type neutron beamforming apparatus for neutron capture treatment according to the present invention has the effect of minimizing the dose contamination value (physical dose value/epithermal neutron value) in BNCT recommended by IAEA-TECDOC-1223.

### DESCRIPTION OF DRAWINGS

FIG. 1 is a conceptual diagram of a neutron capture therapy device.

FIG. 2 is a conceptual diagram showing a beamforming apparatus and target according to the present invention.

FIG. 3 shows the results of computational simulation of neutrons in a conventional beamforming apparatus and a beamforming apparatus according to the present invention.

FIGS. 4A, 4B, 4C, and 4D show the performance of the neutron shaper at the moderator angle of the beam shaper.

FIG. 5 is a cross-sectional view of a labyrinth-type neutron beamforming apparatus for neutron capture therapy, which is the first embodiment according to the present invention.

FIG. 6 is a cross-sectional view of a labyrinth-type neutron beamforming apparatus for neutron capture therapy, which is a second embodiment according to the present invention.

FIG. 7 is a cross-sectional view of a labyrinth-type neutron beamforming apparatus for neutron capture therapy, which is a third embodiment according to the present invention.

FIG. 8 is a cross-sectional view of a labyrinth-type neutron beamforming apparatus for neutron capture therapy, which is a fourth embodiment according to the present invention.

FIG. 9 is a cross-sectional view of a labyrinth-type neutron beamforming apparatus for neutron capture therapy, which is a fifth embodiment according to the present invention.

FIG. 10 is a cross-sectional view of a labyrinth-type neutron beamforming apparatus for neutron capture therapy, which is a sixth embodiment according to the present invention

### MODE FOR DISCLOSURE

Hereinafter, a labyrinth-type neutron beamforming apparatus for neutron capture therapy according to embodiments of the present disclosure will be described in detail with reference to the attached drawings. In the description of the following embodiments, the name of each component may be referred to as different names in the art. However, if there is functional similarity and identity between components, they can be regarded as equivalent components even if modified embodiments are adopted. Additionally, a symbol is attached to each component for convenience of description. However, content shown in the drawings in which such symbols are written does not limit each component to the scope within the drawings. Likewise, even if an embodiment in which a configuration in a drawing is partially modified is adopted, it can be regarded as an equivalent configuration if there is functional similarity and identity. Further, if a component is recognized as a component that should be included in light of the general level of technicians in the relevant technical field, the description thereof will be omitted.

Below, the terms ‘front direction’ and ‘rear direction’ will be used to describe direction. The front refers to the direction in which the beamforming apparatus faces the accelerator, and the rear is defined and explained as the direction in which the neutrons ultimately reach the patient. According to the above-described direction, neutrons are irradiated from the front side to the beamforming apparatus, pass through the beamforming apparatus, and exit to the rear.

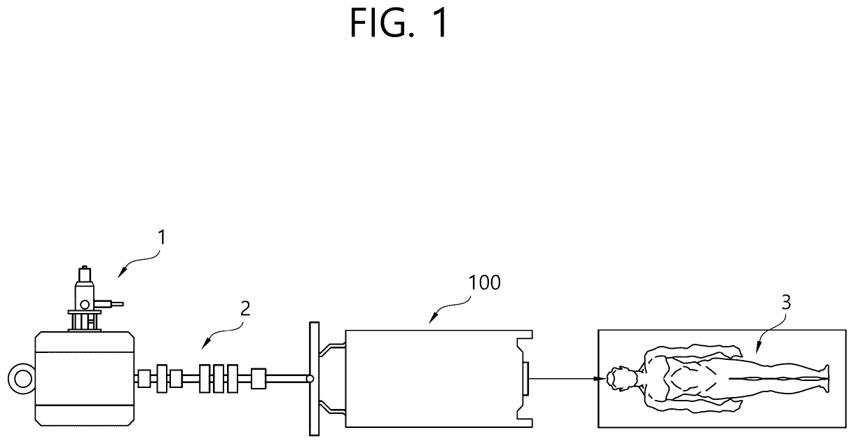

FIG. 1 is a conceptual diagram of a neutron capture therapy device.

As shown, the neutron generator that generates neutrons in boron neutron capture therapy is comprising a particle accelerator (1) such as a cyclotron, linear accelerator, and electrostatic accelerator, an electrostatic accelerator that accelerates the proton beam emitted at high speed from the particle accelerator (1) and a chamber (3) installed on the beam path of the proton beam and provided with a target that collides with the beam and releases neutrons therein.

Neutrons generated from the target can be classified into fast neutrons with an energy of 10 keV or more, epithermal neutrons from 0.5 eV to 10 keV, and thermal neutrons with an energy of 0.5 eV or less. The beamforming apparatus 100 is provided to convert fast neutrons into epithermal neutrons suitable for therapy.

The neutron beam that has passed through the beamforming apparatus 100 is configured to pass through a desired area by a collimator, and is finally irradiated to the affected area of the patient 3 to cause a nuclear reaction.

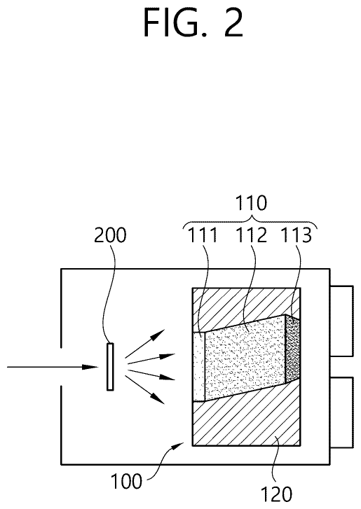

FIG. 2 is a conceptual diagram showing a beamforming apparatus and target according to the present invention.

Referring to FIG. 2, during neutron capture treatment, a particle beam is irradiated to the target 200 to cause a nuclear reaction and also generate gamma rays y. The neutrons and gamma rays generated at this time are emitted at various angles toward the rear. Thereafter,
while passing through the beamforming apparatus 100, the epithermal neutrons are decelerated and filtered into an epithermal neutron region having an appropriate energy and can be irradiated to the patient.

Meanwhile, the beam shape assembly according to the present invention is configured to minimize the reduction of neutron flux and shield gamma rays by adjusting the angle of the beamforming apparatus 100 and the angle of the reflecting surface which is different from the conventional beamforming apparatus 100, which is arranged in a straight line along the direction in which the particle beam is irradiated, that is, the front-to-back direction.

The beamforming apparatus 100 according to the present invention may be configured to include a beam shaping module 110 that determines an area through which neutrons pass and a reflector provided while surrounding the side of the beam shaping module 110.

In one embodiment according to the present invention, the beam shaping module 110 may be provided along an axis extending from the front to the rear. At this time, at least a portion of the beam shaping module 110 may be provided along a path different from the axis.

The beam shaping module 110 may be configured to include a first shield 111, a moderator 112, and a second shield 113. The first shield 111, the moderator 112, and the second shield 113 may be arranged sequentially from the front to the rear.

The first shielding unit 111 is configured to shield fast neutrons. As an example, the first shield 111 may be made of iron or aluminum.

The moderator 112 is configured to slow down neutrons that have passed through the first shield 111 to the outer neutron region. The moderator 112 may include fluorine and be composed of materials such as MgF2, CaF2, PbF2, AlF3, PTFE [(CF2)n], and Fludental (AlF3: 69%, Al: 30%, LiF: 1%).

The second shielding unit 113 is configured to shield thermal neutrons and gamma rays. The second shielding unit 113 may include a thermal neutron filter and a gamma filter.

Thermal neutron filters may be configured to prevent thermal neutrons from passing through. As an example, the thermal neutron filter may include cadmium (cd) or boron and may be configured to have a density of 8.65 g/cm3. Meanwhile, the gamma filter may be configured to prevent gamma rays generated during filtering or deceleration of neutrons from leaking to the collimator. As an example, the gamma filter may include bismuth and may be configured to have a density of 9.75 g/cm3.

The reflector 120 is configured to prevent and shield gamma rays and neutrons from being emitted to unintended areas. The reflector is configured to cover the top, bottom, and both sides of the beam shaping module 110. The reflector 120 may be made of lead or nickel, for example.

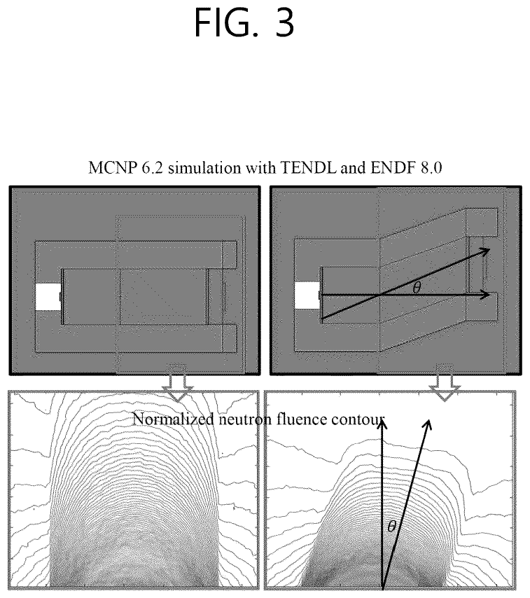

FIG. 3 shows the results of computational simulation of neutrons in a conventional beamforming apparatus and a beamforming apparatus according to the present invention.

Referring to FIG. 3, as a simulation result using Monte Carlo N-Particle code (MCNP, v6.2), the neutron iso-flux distribution is shown, and when an inclined neutron shaping device is used, the direction of neutrons is guided toward the outlet. In other words, it can be confirmed that the neutron flux is maintained.

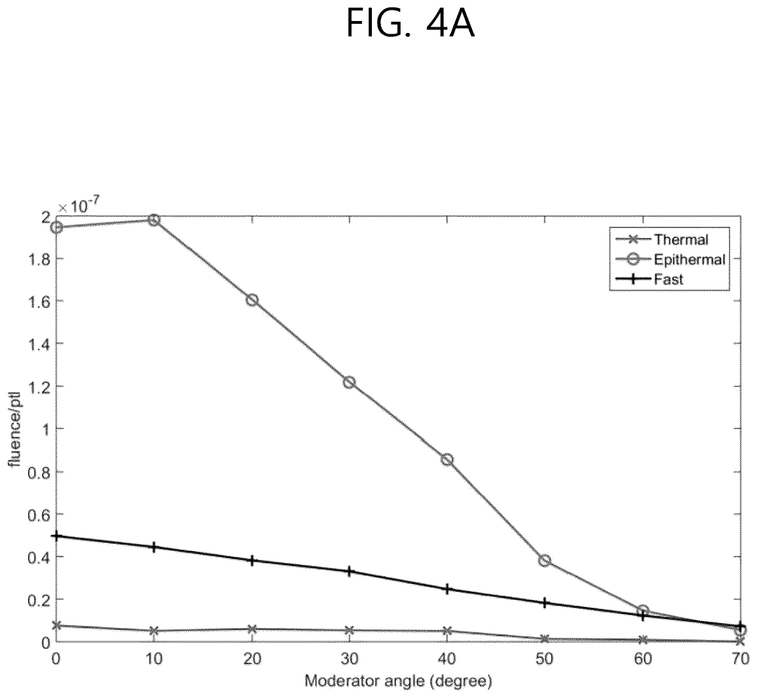

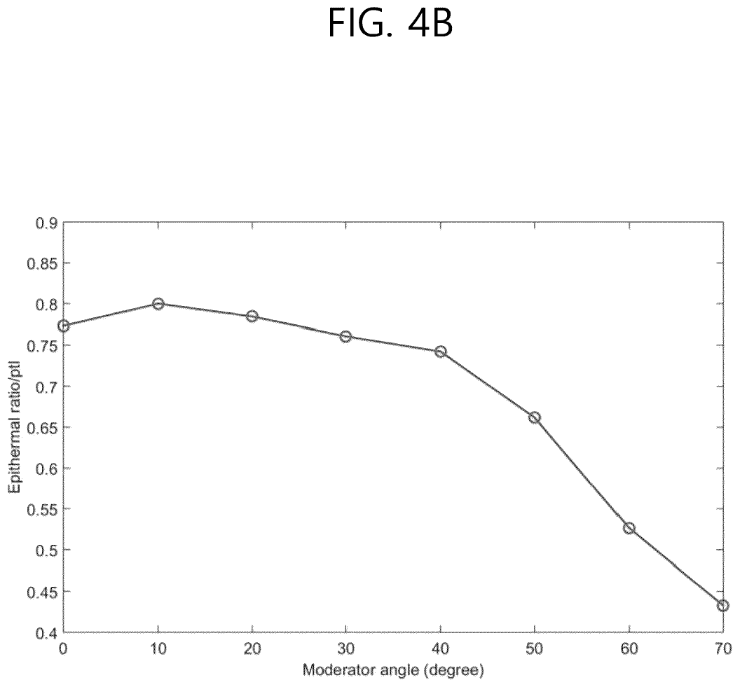

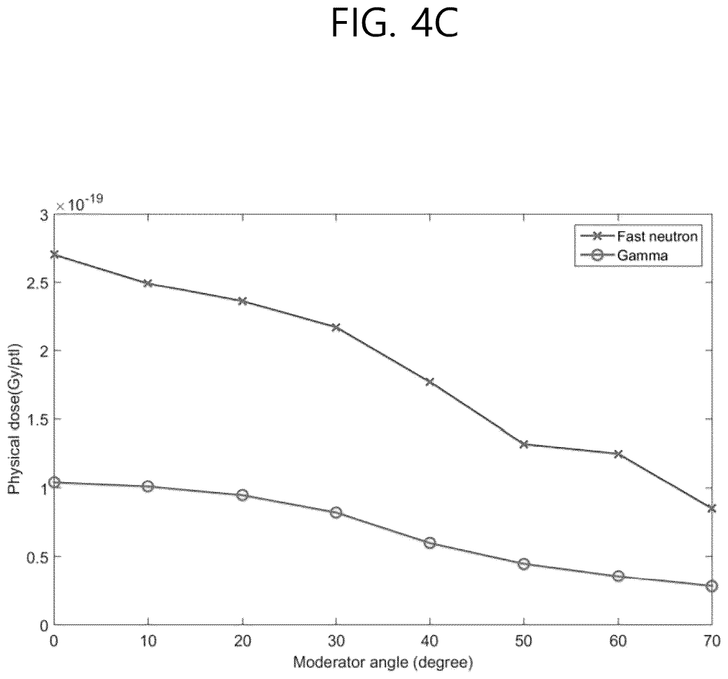

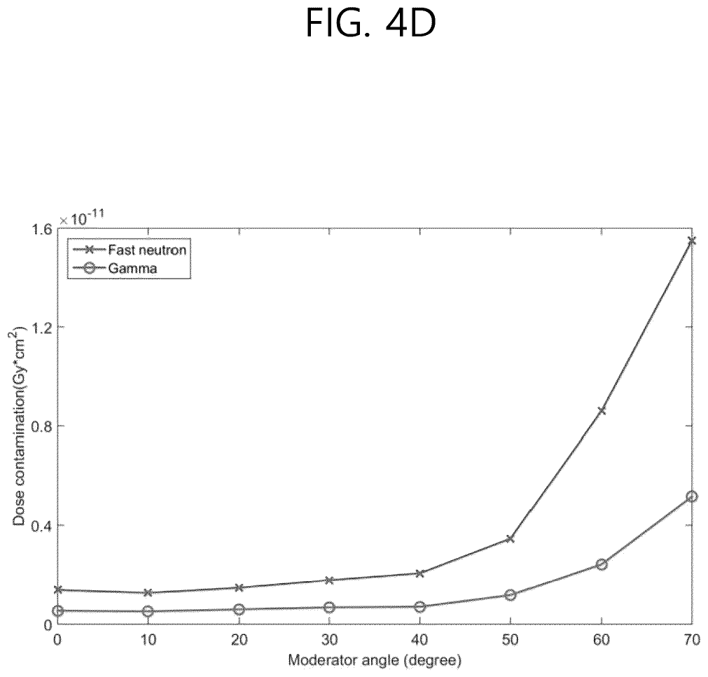

FIGS. 4A, 4B, 4C, and 4D show the performance of the neutron beamforming apparatus at the moderator angle of the beamforming apparatus.

Referring to FIG. 4A, it shows the fluence of each energy of the neutron at the exit of the neutron shaping device according to the moderator angle. It can be seen that the fluence of epithermal neutrons was highest when the moderator angle of the beam shaping module with the front-back axis was around 10 degrees, and that epithermal neutron decreased at angles beyond that.

Referring to FIG. 4B, it can be seen that the epithermal ratio at the exit of the neutron beamforming apparatus according to the present invention is maximum.

Referring to FIG. 4C, it shows the physical dose Gy of fast neutrons and gamma rays depending on the tilt angle of the neutron shaping device. It can be seen that the physical dose Gy of gamma rays decreases as the angle at which the neutron beam shaping module is tilted from the front-to-back axis increases.

Referring to FIG. 4D, this is dose contamination defined by the IAEA. The dose pollution degree is the physical dose Gy due to fast neutrons and gamma rays divided by the epithermal neutron flux at the exit. When the angle (moderator angle) formed by the beam shaping module with the anteroposterior axis is 0 to 40 degrees, the dose contamination level was maintained at a certain level, but it can be seen that it tends to increase after 40 degrees. In FIG. 4C, it can be seen that as the moderator angle of the beam shaping module increases from the anteroposterior axis, the physical dose Gy decreases, but the dose contamination also increases because the flux of epithermal neutrons decreases.

Ultimately, the moderator angle formed by the beam shaping module with the front-to-back axis can maintain the epithermal neutron ratio at an appropriate level within 0 to 40 degrees, and at this time, gamma ray shielding is also properly achieved to maintain low dose contamination.

Referring to FIGS. 3 and 4, in the embodiment of the present invention, when changing the path of the beam shaping module, neutrons required for treatment are guided to the outlet of the beamforming apparatus. Simultaneously, the change in epithermal neutron flux depending on the angle is minimized, and the dose contamination caused by gamma rays and fast neutrons is also minimized.

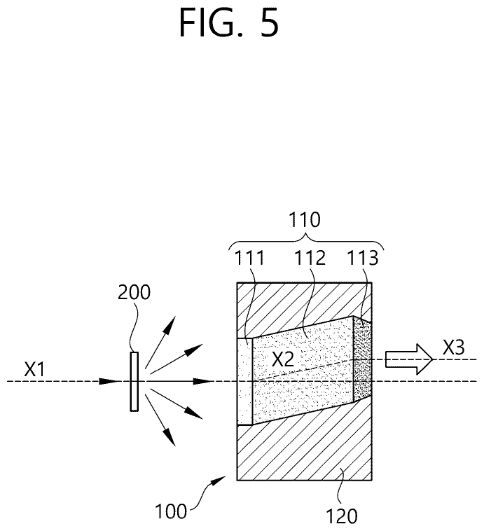

Hereinafter, a modified example of the beamforming apparatus according to the present invention will be described in detail with reference to FIGS. 5 to 10. 5 to 10 conceptually show a state in which the target and the beamforming apparatus are cut along a plane parallel to the horizontal for convenience of explanation.

FIG. 5 is a cross-sectional view of the labyrinth-type neutron beamforming apparatus for neutron capture therapy, which is the first embodiment according to the present invention.

Referring to FIG. 5, in the labyrinth-type neutron beamforming apparatus for neutron capture therapy, which is the first embodiment according to the present invention, the first shielding portion 111 and the second shielding portion 113 are arranged parallel to each other but offset. Meanwhile, the moderator 112 is formed to extend at a certain angle from the front-back axis (x1, the same as the irradiation path of the particle beam). At this time, the second shield 113 may be determined at a position where the front-back axis x1 does not deviate to the outside.

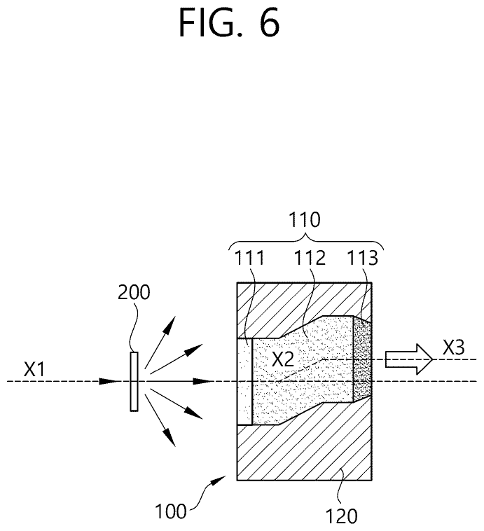

FIG. 6 is a cross-sectional view of a labyrinth-type neutron beamforming apparatus for neutron capture therapy, which is a second embodiment according to the present invention.

Referring to FIG. 6, unlike the first embodiment, the moderator 112 may be divided into a first region, a second region, and a third region. The first area and the third area may be arranged along a direction parallel to the front-to-back axis x1. Meanwhile, the second area may be arranged at a predetermined angle from the front-to-back axis x1. At this time, the reflecting surface formed at the boundary between the reflector 120 and the beam shaping module 110 may be arranged at two angles. That is, it may include a reflective surface parallel to the front-to-back axis and a reflective surface provided at a certain angle to the front-to-back axis. In this embodiment as well, the second shield 113 may be provided in a position through which the axis in the front-back direction can pass.

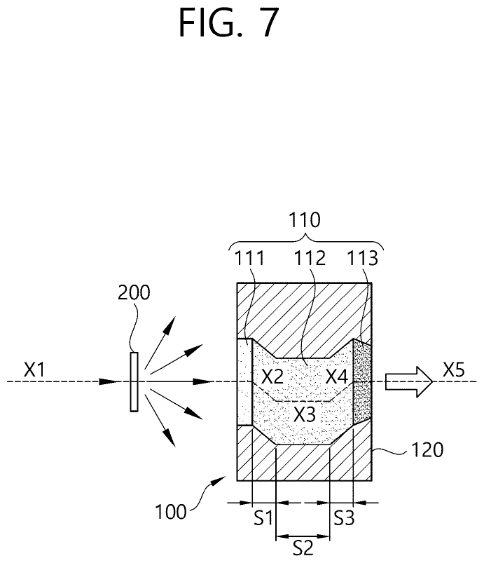

FIG. 7 is a cross-sectional view of a labyrinth-type neutron beamforming apparatus for neutron capture therapy, which is a third embodiment according to the present invention.

Referring to FIG. 7, unlike the second embodiment, the first shielding part 111 and the second shielding part 113 are arranged along the axis x1 in the front-back direction. And the interior of the moderator 112 may be configured to extend along three axes.

The moderator 112 may consist of three regions: a first region, a second region, and a third region. The first region extends along the inclined axis x2 from the point where it is connected to the first shield 111. The second region extends along an axis x3 parallel to the anteroposterior axis x1, while the third region extends along an axis x4, which again extends toward the anteroposterior axis. At this time, the reflective surface of the reflector 120 may form an inclined surface inclined at a positive angle and an inclined surface inclined at a negative angle based on a plane parallel to the front-back direction.

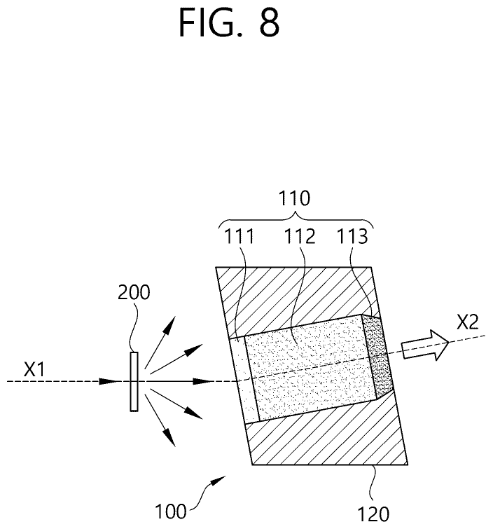

FIG. 8 is a cross-sectional view of a labyrinth-type neutron beamforming apparatus for neutron capture therapy, which is a fourth embodiment according to the present invention.

Referring to FIG. 8, in the fourth embodiment, the beamforming apparatus is configured in the shape of a diamond, and the first shield 111 may be provided at a predetermined angle rather than being perpendicular to the axis x1 in the front-back direction. At this time, the second shield 113 may be provided at a position through which the front-back axis x1 passes. Meanwhile, the first shield 111, the moderator 112, and the second shield 113 of the beam shaping module 100 may be arranged in a straight line along the axis x2, where the axis x2 is forward and backward which is formed at a predetermined angle with the axis of direction (x1). At this time, the inclined surface may be formed at the boundary of the first shield 111, the moderator 112, and the second shield 113.

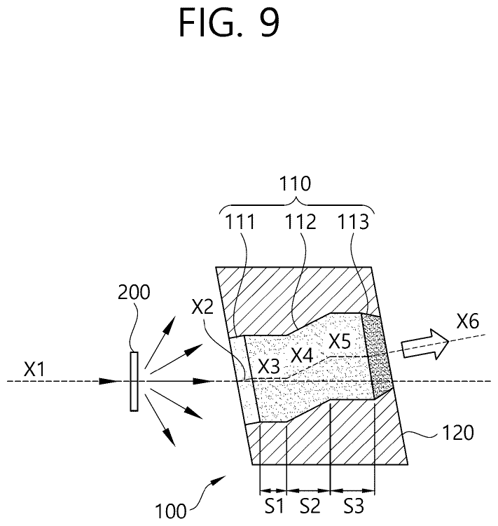

FIG. 9 is a cross-sectional view of a labyrinth-type neutron beamforming apparatus for neutron capture therapy, which is a fifth embodiment according to the present invention.

Referring to FIG. 9, the fifth embodiment may be divided into three areas (S1, S2, and S3) similar to the configuration of the moderator 112 in FIG. 2. Meanwhile, the first shielding part 111 and the second shielding part 113 may be parallel to each other, but may be provided in a direction that is not perpendicular to the front-back axis x1. At this time, the beam shaping module 100 may be provided with a path adjusted along five axes (x2, x3, x4, x5, x6). At this time, the inclined surface may be formed at the boundary of the second area S2 among the first shielding part 111, the second shielding part 113, and the moderator 112.

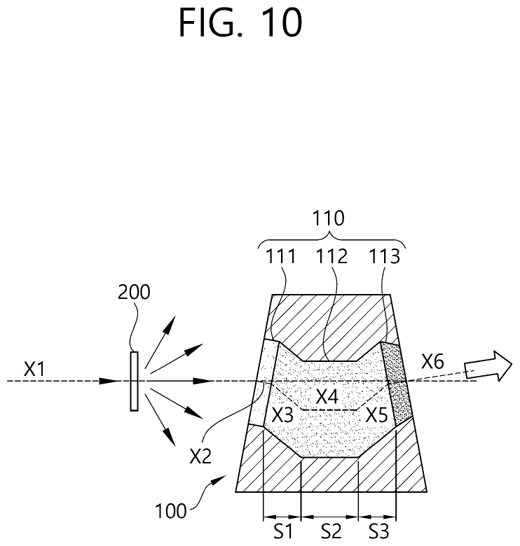

FIG. 10 is a cross-sectional view of a labyrinth-type neutron beamforming apparatus for neutron capture therapy, which is a sixth embodiment according to the present invention.

In the sixth embodiment, unlike the above-described embodiments, the first shielding part 111 and the second shielding part 113 may be arranged not parallel to each other. Additionally, the beamforming apparatus may be formed overall into a trapezoidal shape.

In this embodiment, the first shield 111 is disposed along the front-to-back axis x1 and the inclined axis x2, and the moderator 112 may extend in sections along the three axes. At this time, the moderator 112 may be configured similarly to when divided into three regions (S1, S2, and S3) in the third embodiment. Also, unlike in the third embodiment, the first shield 111 is arranged at an angle adjusted clockwise in FIG. 10 from the front-back axis x1. Additionally, the second shield 113 may be arranged with an angle adjusted counterclockwise from the front-back axis x1.

As described above, the labyrinth-type neutron beamforming apparatus for neutron capture therapy according to the present invention can be provided by changing the path of the beamforming apparatus like a maze and setting the angle of the reflecting surface at various angles from the axis in the forward and backward directions. Therefore, it has the effect of maintaining the neutron flux and dramatically reducing the gamma-ray flux.

1. A labyrinth-type neutron beamforming apparatus for neutron capture therapy comprising;
a neutron beam shaping module is provided along the direction of movement of neutrons and is configured to reduce gamma rays and fast neutron flux and minimize the reduction of epithermal neutron flux; and
a reflector provided surrounding the neutron beam shaping module, wherein the neutron beam shaping module is provided along an axis extending from the front to the rear, and at least a portion of the neutron beam shaping module is provided along a path different from the axis.

2. The labyrinth-type neutron beamforming apparatus for neutron capture therapy according to claim 1,
wherein the neutron beam shaping module is comprising;
a first shield configured to shield the fast neutrons and disposed adjacent to the neutron generation target;
a moderator provided at the rear end of the first shield and configured to reduce the energy of neutrons; and
a second shielding unit provided at the rear end of the moderator and configured to shield thermal neutrons and gamma rays.

3. The labyrinth-type neutron beamforming apparatus for neutron capture therapy according to claim 2, wherein the inclined surface is formed at a boundary of at least one of the first shield, the moderator, and the second shield.

4. The labyrinth-type neutron beamforming apparatus for neutron capture therapy according to claim 3, wherein the reflecting surfaces provided on the left and right sides of the neutron beam shaping module are asymmetrical.

5. The labyrinth-type neutron beamforming apparatus for neutron capture therapy according to claim 4, wherein the inclined surface is formed at the interface between the moderator and the reflector and is provided with at least two surfaces with different angles.

6. The labyrinth-type neutron beamforming apparatus for neutron capture therapy according to claim 5, wherein the two inclined surfaces are disposed at positive and negative angles from a plane parallel to the direction of movement of the particle beam.

7. The labyrinth-type neutron beamforming apparatus for neutron capture therapy according to claim 6, wherein the interface between the moderator and the reflector includes a reflecting surface provided at an angle parallel to the irradiation direction of the particle beam.

8. The labyrinth-type neutron beamforming apparatus for neutron capture therapy according to claim 4, wherein the first shielding unit and the second shielding unit are provided in parallel with each other.

9. The labyrinth-type neutron beamforming apparatus for neutron capture therapy according to claim 8, wherein the center of the first shield and the second shield is located on an irradiation path of the particle beam.

10. The labyrinth-type neutron beamforming apparatus for neutron capture therapy according to claim 8, wherein the center of the first shield and the second shield is located at a different point from the irradiation path of the particle beam.

11. The labyrinth-type neutron beamforming apparatus for neutron capture therapy according to claim 4, wherein the first shielding unit and the second shielding unit are provided at different angles.

12. The labyrinth-type neutron beamforming apparatus for neutron capture therapy according to claim 4, wherein the first shielding agent is disposed not perpendicular to the irradiation direction of the particle beam.
